(19) United States
(12) Patent Application Publication
(10) Pub. No.: US 2026/0053530 A1
(43) Pub. Date: Feb. 26, 2026

(54) INSTRUMENT KIT FOR TRANSOSSEOUS UPPER HYPOGASTRIC PLEXUS NEUROLYTIC BLOCKADE

(71) Applicant: André Luiz Loyelo BARCELLOS, Rio de Janeiro (BR)

(72) Inventor: André Luiz Loyelo BARCELLOS, Rio de Janeiro (BR)

(21) Appl. No.: 19/305,860

(22) Filed: Aug. 21, 2025

(30) Foreign Application Priority Data
Aug. 22, 2024 (BR) 10 2024 017272 8

(51) Int. Cl. A61B 17/34; A61B 90/00; A61K 9/00

(52) U.S. Cl. CPC A61B 17/3472; A61B 17/3496; A61K 9/0019; A61B 2090/3966

(57) ABSTRACT

This is an instrument kit for transosseous superior hypogastric plexus neurolytic blockade characterized by containing a bone transfixation cannula (1), a probe (6) for obstructing the lumen of the bone transfixation cannula (1), a probe (9) for checking the positioning of the bone transfixation cannula (1), a needle (14) for infusion of the neurolytic agent, and a syringe containing a neurolytic agent.

### APPLICATION FIELD

The present invention applies to the field of medicine, more specifically in the treatment of pain conditions or pathologies in the superior hypogastric plexus.

### STATE OF THE ART

Neurolytic blockade of the superior hypogastric plexus is used to treat severe pelvic pain, especially in oncological cases of the pelvis, as well as in difficult-to-treat non-oncological pathologies in gynecology.

Currently, we have two main techniques for performing neurolytic blockade of the superior hypogastric plexus:

### 1^{a} Technique—Retroperitoneal Access (Paravertebral)

Described by Plancarte (Plancarte r, amescua c, patt rb, aldrete ja. superior hypogastric plexus block for pelvic cancer pain. anesthesiology, 1990; 73(2): 236-9) in 1990, uses a 22 gauge needle to penetrate the retroperitoneal space, through the lateral body of the L5 vertebra, with the aim of reaching the anterior part of the body of L5 to perform neurolytic blockade of the superior hypogastric plexus.

This technique, still used, has some restrictions due to anatomical changes in the pelvis and changes in the axial axis, mainly L5 S1, which make it difficult to perform and, in some cases, impossible to perform.

In addition to the anatomical limitations described above, this technique has the potential to injure important structures such as the ureter, nerve roots, especially the L5 root, as well as vascular structures of the pelvis.

### 2^{nd} Technique—Transdiscal Access L5-S1

Described in 2005 by Gurkan Tucker (Turker G, basagan-mogol e, gurbet a, oztuk c, uckunkaya n, sahin s. a new technique for superior hypogastric plexus block: the posteromedian transdiscal approach. tohoku j exp med 2005; 206: 277-81), it is the most widely used technique today. It came as an alternative for performing neurolytic blockade of the superior hypogastric plexus when vertebral and pelvic anatomical difficulties did not allow the classic technique (retroperitoneal) and when the patient had a contraindication to staying in the prone position, since this can be performed with the patient on their side.

This technique is also performed using a 22-gauge needle, with access through the L5-S1 intervertebral disc, projecting the needle to the anterior part of the disc where the neurolytic blockade of the superior hypogastric plexus is performed.

Some complications have also been reported with this technique, the main ones being: infections of the L5 disc (lumbar discitis), root injury, mainly to the L5 root, injury to the dural sac, and pelvic vascular structures.

In addition to the technical difficulties inherent to the procedures described above, we still have a portion of patients who could not be covered by either technique. Cases such as lumbar degenerative process L5-S1 (high prevalence in the population), pathologies of the axial axis compromising the L5-S1 level, such as spondylolisthesis, ankylosing spondylitis, transverse megapophysis of L5, among other osteoarticular pathologies would make it impossible to perform this procedure.

### SUMMARY OF THE INVENTION

In order to solve the problems of the state of the art mentioned above, the instrument kit of the present invention was developed to perform the new technique called transosseous superior hypogastric plexus neurolytic blockade.

The objective to be achieved with the kit and the new technique is to perform the blockade of the superior hypogastric plexus through the body of the first sacral vertebra (transosseous) as an alternative to the techniques described previously. With the possibility of performing it in patients with difficult-to-control tumoral or non-tumorous gynecological pelvic pain.

This technique can be performed on patients with pelvic or vertebral anatomical alterations, as well as on patients with pathologies associated with the L5-S1 segment such as degenerative disc disease L5-S1, spondylolisthesis L5-S1, patients undergoing surgical treatment in the L5-S1 segment, among other musculoskeletal pathologies that are quite common in the general population, and which would be very difficult or unfeasible using the traditional techniques available.

In addition to allowing the superior hypogastric plexus block to be performed in a wider range of patients, the kit of the present invention used in the new technique minimizes the complications described in previous techniques such as ureter injury, nerve root injury, mainly the L5 root, dural sac injury, vascular injury and the dreaded L5-S1 disc infection.

This technique is performed through the S1 vertebral body, penetrating laterally to the S1 pedicle, transfixing the S1 body until it surpasses the anterior cortex of the S1 body where the superior hypogastric plexus is located. In addition to allowing use in patients who have difficulty performing the previous techniques, it can be performed in patients with axial or pelvic osteoarticular pathologies, in addition to avoiding the most frequent complications of the previous techniques.

### DESCRIPTION OF DRAWINGS

FIG. 1 illustrates a perspective view of the bone transfixation cannula (1) composed of a handle (2) made of plastic material connected to a hollow stainless steel cannula (3) 200 to 300 mm, preferably 250 mm, long and 4 mm thick, equipped with a tip (4) beveled at 40 degrees to facilitate the passage of the cannula (3) through the bone tissue. In the upper part of the handle (2) there is a cavity that allows access to the cannula (3). At the upper end of the cannula (1) is the handle (5) of the probe (6) for obstructing the lumen of the bone transfixation cannula (1). The probe (6) is fitted to the bone transfixation cannula (1) by means of the cavity of the handle (2).

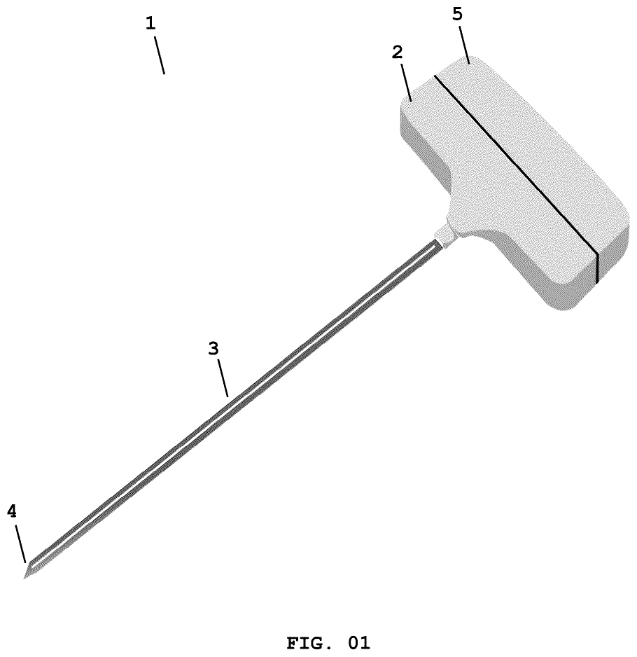

FIG. 2 illustrates a perspective view of the probe (6) for obstructing the lumen of the bone transfixation cannula (1). The probe (6) consists of a handle (5) made of plastic material connected to a solid stainless steel cannula (7) measuring 205 to 305 mm, preferably 255 mm, in length, sufficient for its tip (8) to align with the tip (4) of the bone transfixation cannula (1) and with a thickness one millimeter smaller than the internal diameter of the cannula (3). The cannula (7) has a tip (8) beveled at 40 degrees, following the tip (4) of the bone transfixation cannula (1). When the probe (6) is fitted into the bone transfixation cannula (1), the solid cannula (7) increases the resistance of the hollow cannula (3) and the fitted handles (2) and (5) serve as support for introducing the bone transfixation cannula (1) into the bone tissue.

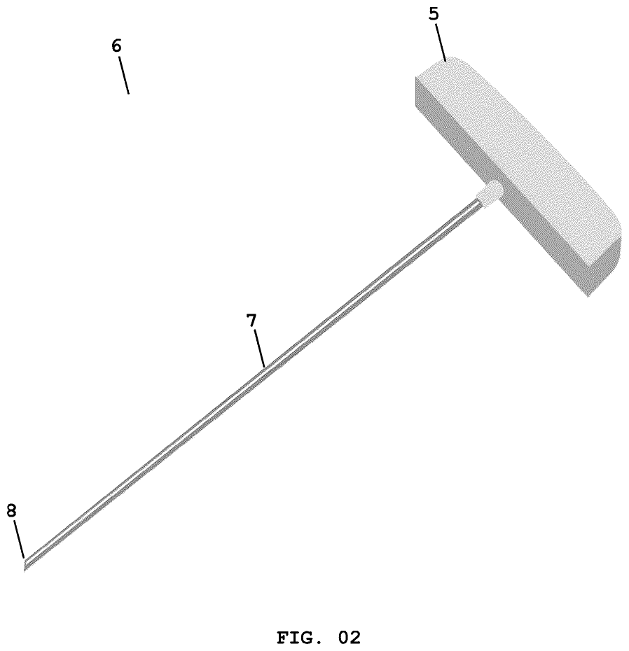

FIG. 3 illustrates a perspective view of the probe (9) for checking the positioning of the bone transfixation cannula. The probe (9) is formed at its upper end by a radiopaque blunt tip (10) connected to a solid cannula (11) with a flat tip (12). The cannula (11) is equipped with a safety ring (13). The length between the safety ring (13) and the tip (12) of the cannula (11) is 205 to 305 mm, preferably 255 mm, being 5 mm more than the length of the cannula (3), ensuring a safe area for carrying out the procedure for checking the exact position of the bone transfixation cannula (1) under radioscopy.

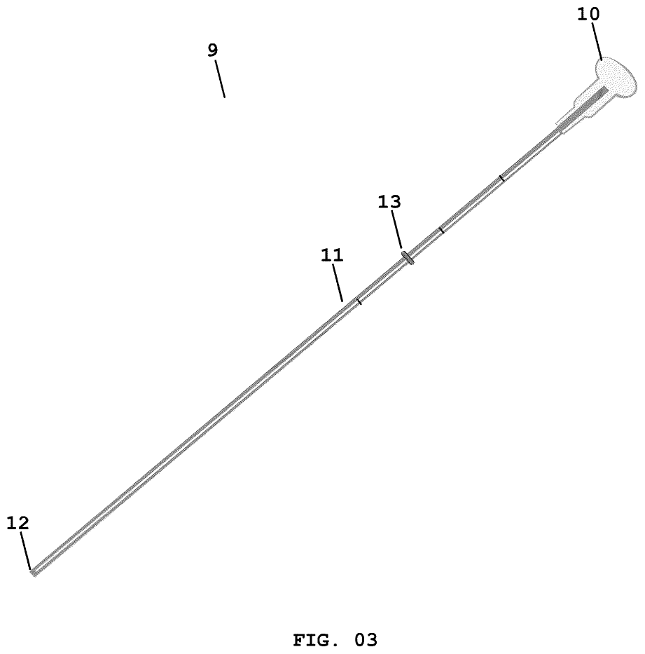

FIG. 4 illustrates a perspective view of the needle (14) for infusion of the 22 gauge neurolytic agent with a body (18) measuring 205 to 305 mm, preferably 255 mm, in length, where the body (18) of the neurolytic agent infusion needle is 5 mm longer than the bone transfixation cannula (1). The needle (14), at its upper end, is equipped with a connector (15) for a syringe connected to a hose (16) that is connected to the connector (17) to hold the body of the needle (18). The syringe is equipped with the neurolytic agent that is conducted by the hose (16), allowing the infusion to be distant from the site where radiation will be emitted to control the procedure, reducing the action of the radiation on the hand of the professional performing the procedure.

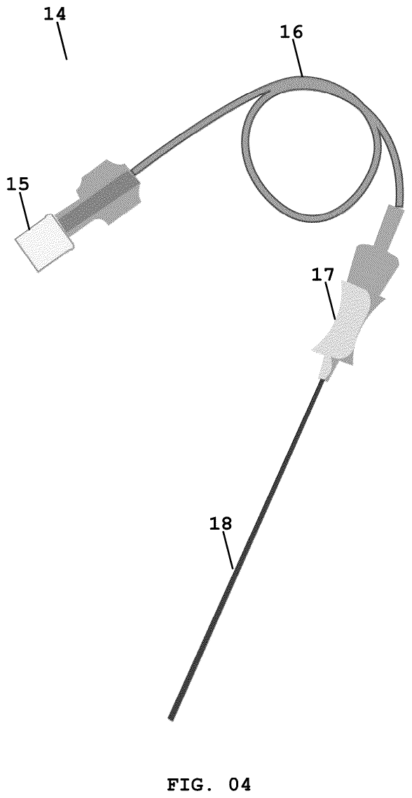

FIG. 5 illustrates a radiograph with a patient positioned on the surgical table with the radioscopic device in the true anteroposterior position for visualization of the S1 pedicle. The black arrowhead indicates the placement point of the bone transfixation cannula (1) on the lateral border inferior to the S1 pedicle at a 30 degrees lateral angle.

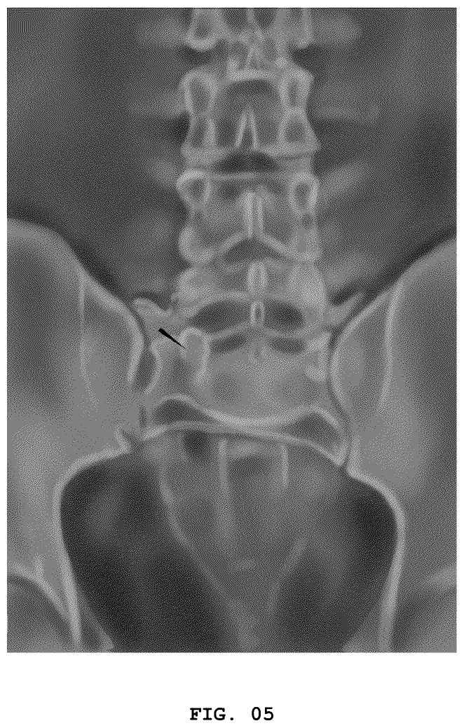

FIG. 6 illustrates a radiograph with a lateral view of the image in FIG. 5. Under the control of the radioscope in profile incidence, the bone transfixion cannula (1) is introduced parallel to the vertebral plateau of S1 until it breaks the cortex of the body of S1. The black rectangle represents the path taken by the bone transfixion cannula (1) in S1 until it reaches the hypogastric plexus.

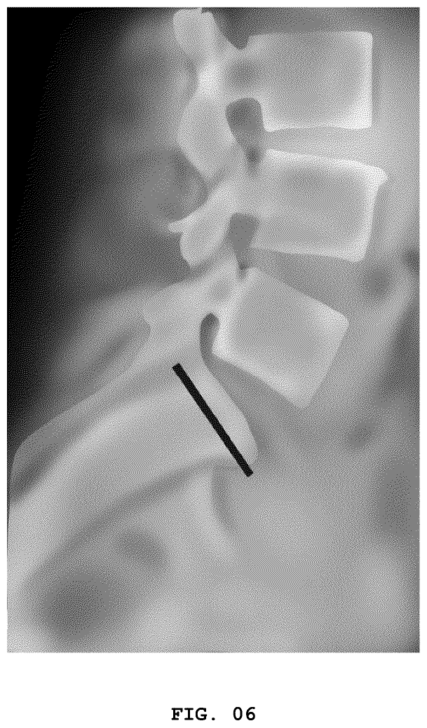

FIG. 7 illustrates a radiograph, again with the patient positioned on the surgical table with the radioscopic device in the anteroposterior position, to check the positioning of the bone transfixion cannula (1) which should be before the midline of the body of S1. The black arrow bridge represents the position of the tip of the bone transfixion cannula (1).

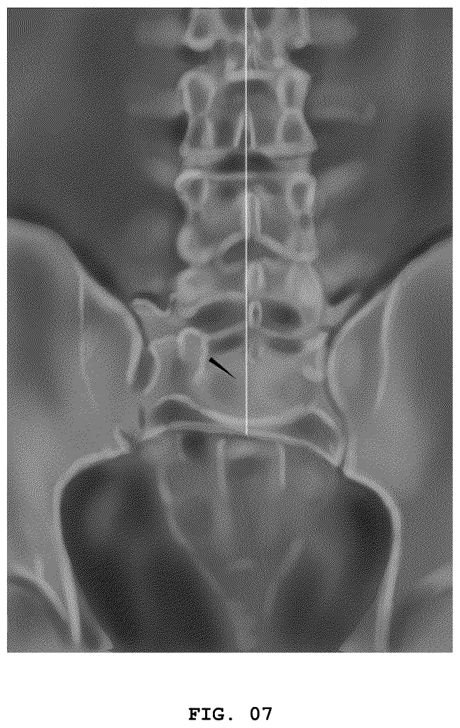

FIG. 8 illustrates an X-ray, again with the radioscopic device in profile position with a side view of the image in FIG. 5. It illustrates the introduction, through the bone transfixation cannula (1), of the probe (9) to check for rupture of the anterior cortex of S1 and its adequate positioning not exceeding 5 mm from the cortex as a safety measure guaranteed by the retention ring (13). After satisfactory radioscopic control, it is possible to remove the probe (9) from the bone transfixation cannula (1) and introduce the infusion needle (18) and inject the neurolytic agent, proceeding to block the superior hypogastric plexus.

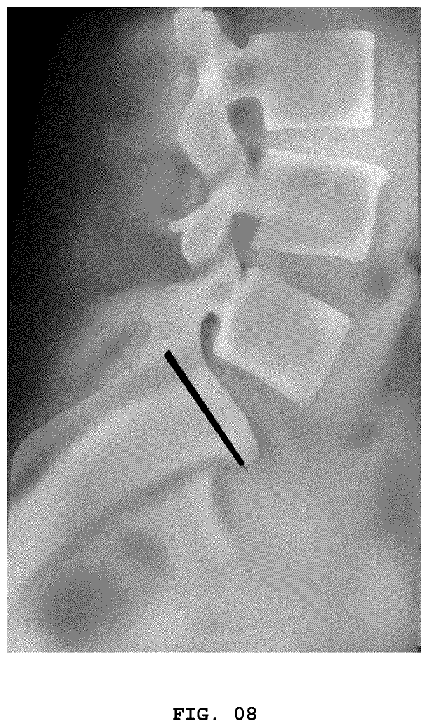

### DETAILED DESCRIPTION OF THE INVENTION

The instrument kit for transosseous superior hypogastric plexus neurolytic block of the present invention is characterized by containing:
a bone transfixation cannula (1) consisting of a handle (2) made of plastic material connected to a hollow stainless steel cannula (3) 200 to 300 mm, preferably 250 mm, in length and 4 mm in thickness equipped with a tip (4) beveled at 40 degrees that perforates the bone tissue, in the upper part of the handle (2) there is a cavity that allows access to the cannula (3), in the upper end of the cannula (1) there is a handle (5) of the probe (6) for obstructing the lumen of the bone transfixation cannula (1) that is fitted into the bone transfixation cannula (1) by means of the cavity of the handle (2);
a probe (6) for obstructing the lumen of the bone transfixation cannula (1), consisting of a handle (5) made of plastic material connected to a solid stainless steel cannula (7) measuring 205 to 305 mm, preferably 255 mm in length, allowing its tip (8) to align with the tip (4) of the bone transfixation cannula (1) and with a thickness that is millimetrically smaller than the internal diameter of the cannula (3), the cannula (7) is equipped with a tip (8) beveled at 40 degrees that aligns with the tip (4) of the bone transfixation cannula (1) and, when the probe (6) is fitted to the bone transfixation cannula (1), the solid cannula (7) increases the resistance of the hollow cannula (3) and the handles (2) and (5) fit together and serve as support for the introduction of the bone transfixation cannula (1) into the tissue bone;
a probe (9) for checking the positioning of the bone transfixation cannula formed at its upper end by a radiopaque blunt tip (10) connected to a solid cannula (11) with a flat tip (12), the cannula (11) is equipped with a safety ring (13), the length between the safety ring (13) and the tip (12) of the cannula (11) is 205 to 305 mm, preferably 255 mm, being 5 mm more than the length of the cannula (3) ensuring a safe area for carrying out the procedure for checking the exact position of the bone transfixation cannula (1) under radioscopy, and the length between the safety ring (13) and the radiopaque blunt tip (10) of the cannula (11) is 70 to 80 mm;
a 22 gauge needle (14) for infusion of the neurolytic agent with a body (18) 205 to 305 mm, preferably 255 mm, in length, where the body (18) of the neurolytic agent infusion needle, made of stainless steel, is 5 mm longer than the bone transfixation cannula (1), the needle (14), at its upper end, is equipped with a connector (15) for a syringe connected to a hose (16) 200 to 300 mm in length that is connected to the connector (17) for manipulating the needle (18); and
a syringe containing a neurolytic agent that is guided through the hose (16) at a safe distance from the site where radiation will be emitted to control the procedure, reducing the action of radiation on the hand of the professional performing the procedure.

The transosseous superior hypogastric block procedure performed using the specific kit of the present invention follows the following steps:
1—With the patient positioned on the surgical table, position a radioscopic device in the anteroposterior position to view the S1 pedicle;
2—Introduce the tip of the bone transfixation cannula (1) into the lower lateral edge of the S1 pedicle at a 30-degree lateral angle;
2—Position the radioscope in profile view and continue introducing the bone transfixation cannula (1) parallel to the S1 vertebral plateau until it breaks the cortex of the S1 body and reaches the hypogastric plexus;
3—Position the radioscopic device in the anteroposterior position and check whether the positioning of the bone transfixation cannula (1) is before the midline of the S1 body;
4—Remove the probe (6) obstructing the lumen of the bone transfixation cannula (1) by pulling it by the handle (5);
5—Position the radioscopic device in profile view with a lateral view;
6—Introduce the probe (9) through the bone transfixation cannula (1) until it reaches the retention ring (13) and check whether the anterior cortex of S1 has been ruptured and whether its positioning has not exceeded 5 mm from the cortex;
7—Once the correct position of the bone transfixation cannula (1) has been confirmed, remove the probe (9) from the bone transfixation cannula (1);
8—Introduce the infusion needle (18) from the bone transfixation cannula (1); and
9—Inject the neurolytic agent, blocking the superior hypogastric plexus.

1. Instrument kit for transosseous superior hypogastric plexus neurolytic blockade characterized by containing
a bone transfixation cannula (1) composed of a handle (2) connected to a hollow cannula (3) equipped with a tip (4) piercing the bone tissue, in the upper part of the handle (2) there is a cavity that allows access to the cannula (3) where the handle (5) of the probe (6) for obstructing the lumen of the bone transfixation cannula (1) fits;
a probe (6) for obstructing the lumen of the bone transfixation cannula (1), consisting of a handle (5) connected to a solid cannula (7), with a smaller diameter than that of the cannula (3), equipped with a beveled tip (8) that aligns with the tip (4) of the bone transfixation cannula (1), when the probe (6) is fitted to the bone transfixation cannula (1), the solid cannula (7) increases the resistance of the hollow cannula (3) and the handles (2) and (5) fit together and serve as support for the introduction of the bone transfixation cannula (1) into the bone tissue;
a probe (9) for checking the positioning of the bone transfixation cannula (1) formed at its upper end by a blunt radiopaque tip (10) connected to a solid cannula (11) equipped with a safety ring (13) and with a flat tip (12);
a needle (14) for infusing the neurolytic agent with a body (18) equipped, at its upper end, with a connector (15) for a syringe connected to a hose (16) connected to the connector (17) for manipulating the needle (18);
and a syringe containing a neurolytic agent.

2. Kit, according to claim 1, characterized by the fact that the handle (2) of the bone transfixation cannula (1) is made of plastic material.

3. Kit, according to claim 1, characterized by the fact that the cannula (3) of the bone transfixation cannula (1) is made of stainless steel.

4. Kit according to claim 1, characterized by the fact that the cannula (3) of the bone transfixation cannula (1) is 200 to 300 mm, preferably 250 mm, long and 4 mm thick, and its tip (4) is beveled at 40 degrees.

5. Kit according to claim 1, characterized by the fact that the handle (5) of the probe (6) is made of plastic.

6. Kit according to claim 1, characterized by the fact that the cannula (7) of the probe (6) is solid and made of stainless steel, 205 to 305 mm, preferably 255 mm long.

7. Kit according to claim 1, characterized by the fact that the cannula (7) of the probe (6) has a thickness that is millimetrically smaller than the internal diameter of the cannula (3).

8. Kit, according to claim 1, characterized by the fact that the tip (8) of the cannula (7) of the probe (6) is beveled at 40 degrees.

9. Kit, according to claim 1, characterized by the fact that the probe (9) has a length between the safety ring (13) and the tip (12) of the cannula (11) of 205 to 305 mm, preferably 255 mm, being 5 mm more than the length of the cannula (3), and the length between the safety ring (13) and the radiopaque blunt tip (10) of the cannula (11) is 70 to 80 mm.

10. Kit, according to claim 1, characterized by the fact that the needle (14) for infusion of the neurolytic agent is 22 gauge and its body (18) is made of stainless steel and is 205 to 305 mm, preferably 255 mm, in length and its length is 5 mm greater than the bone transfixation cannula (1).

11. Kit according to claim 1, characterized by the fact that the hose (16) of the needle (14) is 200 to 300 mm long.
